SEQUENCE LISTING (4 sequences)

SEQ ID NO: 1 - DNA, 1350 bp, Corynebacterium glutamicum
  CDS (233)..(1123)
ctctttacctggcaaaaacacagcgatcggatgcgggtcaaaggtcaccatgacacaaggcacacccagctcctcggcctgcttcttggcctcgccgattaaactttgatgcccccggtggagcccatcaaacacaccaatggtgactactgatccttgaagatcagccggaacgctgtctagtccactccaaatatccactgttttagactacggcatagactcaacagacatgaatgctcctgcccctaaacctggactcgtgatcgtcgacaagcccgccggaatgacatcccatgacgtggtgtccaaattgcgccgcgcattttccacccgcaaagtaggccacgcaggcaccctcgaccccatggcaaccggcgtgttagtcgtcggaattgagcgcggaacccgcttcctggcacacatggtggcctccaccaaagcctacgacgccaccattcgactcggcgccgccaccagcaccgatgatgcagaaggcgaggttatctccacaacagacgcatccggcctcgaccacagcaccatccttgctgaaatcgtcaacctcaccggcgacatcatgcaaaaacccaccaaagtctccgccatcaaaatcgacggcaaacgcgcccacgaacgcgtccgcgacggcgaagaagtagacattcccgcacgtcccgtcaccgtcagcgtctttgacgtgctcgactaccacgtcgacggtgaattttatgacttagatgtgcgcgtccactgctcctccggcacctacatccgcgcgctcgcccgcgacctcggcaacgctttgcaggtcggcggccacctgaccgcgcttaggcgcacagaggtcggcccttttacgcttaacgacgcgacccccctctccaaactccaagagaatccagaactctccctcaacctcgaccaggcactcacccgcagttacccagtccttgacatcaccgaagacgaaggcgttgacctgtccatgggcaaatggttggaacctcgcggactgaaaggcgtccacgctgcagtaacaccatcaggaaaagccgtggcgctcatcgaagaaaagggcaaacgcctggccaccgtgtttgttgctcaccccaacactctttagttggtctgccagaagccgatttaagaggtcgaatttcgctagataggtgtgagtgtggaaagagcggtttggagggcttaaacgcgattctgagagggcactatttcaggtgggctgtggaaataaaccaaaaccaactccgccaatccaagccttggtttcaaacgcacatttccagaccagcaaagtgtcattgcagaccaaagacgccccgaaaaaggggc

SEQ ID NO: 2 - protein, 297 aa, Corynebacterium glutamicum
MNAPAPKPGLVIVDKPAGMTSHDVVSKLRRAFSTRKVGHAGTLDPMATGVLVVGIERGTRFLAHMVASTKAYDATIRLGAATSTDDAEGEVISTTDASGLDHSTILAEIVNLTGDIMQKPTKVSAIKIDGKRAHERVRDGEEVDIPARPVTVSVFDVLDYHVDGEFYDLDVRVHCSSGTYIRALARDLGNALQVGGHLTALRRTEVGPFTLNDATPLSKLQENPELSLNLDQALTRSYPVLDITEDEGVDLSMGKWLEPRGLKGVHAAVTPSGKAVALIEEKGKRLATVFVAHPNTL

SEQ ID NO: 3 - DNA, 28 bp, Corynebacterium glutamicum
tgggtaccacggcatagactcaacagac

SEQ ID NO: 4 - DNA, 28 bp, Corynebacterium glutamicum
gatctagacggcttctggcagaccaact

(19) United States
(12) Patent Application Publication
(10) Pub. No.: US 2002/0115160 A1
(43) Pub. Date: Aug. 22, 2002

(54) Nucleotide sequences which code for the truB gene

(76) Inventors: Mike Farwick, Bielefeld (DE); Klaus Huthmacher, Gelnhausen (DE); Walter Pfefferle, Halle (DE); Brigitte Bathe, Salzkotten (DE)

Correspondence Address: SMITH, GAMBRELL & RUSSELL, LLP, SUITE 800, 1850 M STREET, N.W., WASHINGTON, DC, 20036, US

(21) Appl. No.: 09/963,690

(22) Filed: Sep. 27, 2001

(30) Foreign Application Priority Data
Sep. 27, 2000 (DE) 100 47 864.6

(51) Int. Cl. C12P 13/08; C07H 21/04; C12N 9/22; C12P 21/02; C12N 1/21; C12N 15/74

(52) U.S. Cl. 435/115; 435/69.1; 435/199; 435/252.3; 536/23.2; 435/320.1

(57) ABSTRACT

The invention relates to an isolated polynucleotide having a polynucleotide sequence which codes for the truB gene, and a host-vector system having a coryneform host bacterium in which the truB gene is present in attenuated form and a vector which carries at least the truB gene according to SEQ ID No 1, and the use of polynucleotides which comprise the sequences according to the invention as hybridization probes.

### BACKGROUND OF THE INVENTION

The invention provides nucleotide sequences from coryneform bacteria which code for the truB gene and a process for the fermentative preparation of amino acids using bacteria in which the endogenous truB gene is enhanced. All references cited herein are expressly incorporated by reference. Incorporation by reference is also designated by the term “I.B.R.” following any citation.

L-Amino acids, in particular L-lysine, are used in human medicine and in the pharmaceuticals industry, in the foodstuffs industry and very particularly in animal nutrition.

It is known that amino acids are prepared by fermentation from strains of coryneform bacteria, in particular Corynebacterium glutamicum. Because of their great importance, work is constantly being undertaken to improve the preparation processes. Improvements to the process can relate to fermentation measures, such as, for example, stirring and supply of oxygen, or the composition of the nutrient media, such as, for example, the sugar concentration during the fermentation, or the working up to the product form by, for example, ion exchange chromatography, or the intrinsic output properties of the microorganism itself.

Methods of mutagenesis, selection and mutant selection are used to improve the output properties of these microorganisms. Strains which are resistant to antimetabolites or are auxotrophic for metabolites of regulatory importance and produce amino acids are obtained in this manner.

Methods of the recombinant DNA technique have also been employed for some years for improving the strain of Corynebacterium strains which produce L-amino acid, by amplifying individual amino acid biosynthesis genes and investigating the effect on the amino acid production.

The invention provides new measures for improved fermentative preparation of amino acids.

### BRIEF SUMMARY OF THE INVENTION

Where L-amino acids or amino acids are mentioned in the following, this means one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine is particularly preferred.

When L-lysine or lysine are mentioned in the following, not only the bases but also the salts, such as e.g. lysine monohydrochloride or lysine sulfate, are meant by this.

The invention provides an isolated polynucleotide from coryneform bacteria, comprising a polynucleotide sequence which codes for the truB gene, chosen from the group consisting of

a) polynucleotide which is identical to the extent of at least 70% to a polynucleotide which codes for a polypeptide which comprises the amino acid sequence of SEQ ID No. 2,

b) polynucleotide which codes for a polypeptide which comprises an amino acid sequence which is identical to the extent of at least 70% to the amino acid sequence of SEQ ID No. 2,

c) polynucleotide which is complementary to the polynucleotides of a) or b), and

d) polynucleotide comprising at least 15 successive nucleotides of the polynucleotide sequence of a), b) or c),

The polypeptide preferably having the activity of tRNA pseudouridine 55 synthase.

The invention also provides the above-mentioned polynucleotide, this preferably being a DNA which is capable of replication, comprising:

(i) the nucleotide sequence shown in SEQ ID No. 1, or

(ii) at least one sequence which corresponds to sequence (i) within the range of the degeneration of the genetic code, or

(iii) at least one sequence which hybridizes with the sequence complementary to sequence (i) or (ii), and optionally

(iv) sense mutations of neutral function in (i).

The invention also provides

a polynucleotide, in particular DNA, which is capable of replication and comprises the nucleotide sequence as shown in SEQ ID No. 1;

a polynucleotide which codes for a polypeptide which comprises the amino acid sequence as shown in SEQ ID No. 2;

a vector containing the polynucleotide according to the invention, in particular a shuttle vector or plasmid vector, and

coryneform bacteria which contain the vector or in which the endogenous truB gene is enhanced.

The invention also provides polynucleotides, which substantially comprise a polynucleotide sequence, which are obtainable by screening by means of hybridization of a corresponding gene library of a coryneform bacterium, which comprises the complete gene or parts thereof, with a probe which comprises the sequence of the polynucleotide according to the invention according to SEQ ID No. 1 or a fragment thereof, and isolation of the polynucleotide sequence mentioned.

### BRIEF DESCRIPTION OF THE FIGURES

FIG. 1: Map of the plasmid pEC-XK99E

FIG. 2: Map of the plasmid pEC-XK99EtruBex

The abbreviations and designations used have the following meaning:

| | |
|---|---|
| Kan: | Kanamycin resistance gene aph(3′)-IIa from |
| | Escherichia coli |
| HindIII | Cleavage site of the restriction enzyme |
| | HindIII |
| XbaI | Cleavage site of the restriction enzyme XbaI |
| KpnI | Cleavage site of the restriction enzyme KpnI |
| Ptrc | trc promoter |
| T1 | Termination region T1 |
| T2 | Termination region T2 |
| Per | Replication effector per |
| Rep | Replication region rep of the plasmid pGA1 |
| LacIq | lacIq repressor of the lac operon of |
| | Escherichia coil |
| TruB | Cloned truB gene |

### DETAILED DESCRIPTION OF THE INVENTION

Polynucleotides which comprise the sequences according to the invention are suitable as hybridization probes for RNA, cDNA and DNA, in order to isolate, in the full length, nucleic acids or polynucleotides or genes which code for tRNA pseudouridine 55 synthase or to isolate those nucleic acids or polynucleotides or genes which have a high similarity of sequence with that of the truB gene. They can also be attached as a probe to so-called “arrays”, “micro arrays” or “DNA chips” in order to detect and to determine the corresponding polynucleotides or sequences derived therefrom, such as e.g. RNA or cDNA.

Polynucleotides which comprise the sequences according to the invention are furthermore suitable as primers with the aid of which DNA of genes which code for tRNA pseudouridine 55 synthase can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides with a length of at least 31, 32, 33, 34, 35, 36, 37, 38, 39 or 40, or at least 41, 42, 43, 44, 45, 46, 47, 48, 49 or 50 nucleotides are also suitable. Oligonucleotides with a length of at least 100, 150, 200, 250 or 300 nucleotides are optionally also suitable.

“Isolated” means separated out of its natural environment.

“Polynucleotide” in general relates to polyribonucleotides and polydeoxyribonucleotides, it being possible for these to be non-modified RNA or DNA or modified RNA or DNA.

The polynucleotides according to the invention include a polynucleotide according to SEQ ID No. 1 or a fragment prepared therefrom and also those which are at least in particular 70% to 80%, preferably at least 81% to 85%, particularly preferably at least 86% to 90%, and very particularly preferably at least 91%, 93%, 95%, 97% or 99% identical to the polynucleotide according to SEQ ID No. 1 or a fragment prepared therefrom.

“Polypeptides” are understood as meaning peptides or proteins which comprise two or more amino acids bonded via peptide bonds.

The polypeptides according to the invention include a polypeptide according to SEQ ID No. 2, in particular those with the biological activity of tRNA pseudouridine 55 synthase, and also those which are at least 70% to 80%, preferably at least 81% to 85%, particularly preferably at least 86% to 90%, and very particularly preferably at least 91%, 93%, 95%, 97% or 99% identical to the polypeptide according to SEQ ID No. 2 and have the activity mentioned.

The invention furthermore relates to a process for the fermentative preparation of amino acids chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using coryneform bacteria which in particular already produce amino acids and in which the nucleotide sequences which code for the truB gene are enhanced, in particular over-expressed.

The term “enhancement” in this connection describes the increase in the intracellular activity of one or more enzymes in a microorganism which are coded by the corresponding DNA, for example by increasing the number of copies of the gene or allele or of the genes or alleles, using a potent promoter or using a gene or allele which codes for a corresponding enzyme (protein) having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

The microorganisms which the present invention provides can produce L-amino acids from glucose, sucrose, lactose, fructose, maltose, molasses, starch, cellulose or from glycerol and ethanol. They can be representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum (C. glutamicum), are in particular the known wild-type strains

Corynebacterium glutamicum ATCC13032

Corynebacterium acetoglutamicum ATCC15806

Corynebacterium acetoacidophilum ATCC13870

Corynebacterium thermoaminogenes FERM BP-1539

Corynebacterium melassecola ATCC17965

Brevibacterium flavum ATCC14067

Brevibacterium lactofermentum ATCC13869 and

Brevibacterium divaricatum ATCC14020

And L-amino acid-producing mutants or strains prepared therefrom.

The new truB gene from C. glutamicum which codes for the enzyme tRNA pseudouridine 55 synthase (EC 4.2.1.70) has been isolated.

To isolate the truB gene or also other genes of C. glutamicum, a gene library of this microorganism is first set up in Escherichia coli (E. coli). The setting up of gene libraries is described in generally known textbooks and handbooks. The textbook by Winnacker: Gene und Klone, Eine Einführung in die Gentechnologie [Genes and Clones, An Introduction to Genetic Engineering] (Verlag Chemie, Weinheim, Germany, 1990) I.B.R., or the handbook by Sambrook et al.: Molecular Cloning, A Laboratory Manual (Cold Spring Harbor Laboratory Press, 1989) I.B.R. may be mentioned as an example. A well-known gene library is that of the E. coli K-12 strain W3110 set up in λ vectors by Kohara et al. (Cell 50, 495-508 (1987)) I.B.R. Bathe et al. (Molecular and General Genetics, 252:255-265, 1996) I.B.R. describe a gene library of C. glutamicum ATCC13032, which was set up with the aid of the cosmid vector SuperCos I (Wahl et al., 1987, Proceedings of the National Academy of Sciences USA, 84:2160-2164 I.B.R.) in the E. coli K-12 strain NM554 (Raleigh et al., 1988, Nucleic Acids Research 16:1563-1575 I.B.R.).

Börmann et al. (Molecular Microbiology 6(3), 317-326) (1992)) I.B.R. in turn describe a gene library of C. glutamicum ATCC13032 using the cosmid pHC79 (Hohn and Collins, Gene 11, 291-298 (1980)) I.B.R.

To prepare a gene library of C. glutamicum in E. coli it is also possible to use plasmids such as pBR322 (Bolivar, Life Sciences, 25, 807-818 (1979) I.B.R.) or pUC9 (Vieira et al., 1982, Gene, 19:259-268 I.B.R.). Suitable hosts are, in particular, those E. coli strains which are restriction- and recombination-defective. An example of these is the strain DH5αmcr, which has been described by Grant et al. (Proceedings of the National Academy of Sciences USA, 87 (1990) 4645-4649) I.B.R. The long DNA fragments cloned with the aid of cosmids can in turn be subcloned in the usual vectors suitable for sequencing and then sequenced, as is described e.g. by Sanger et al. (Proceedings of the National Academy of Sciences of the United States of America, 74:5463-5467, 1977) I.B.R.

The resulting DNA sequences can then be investigated with known algorithms or sequence analysis programs, such as e.g. that of Staden (Nucleic Acids Research 14, 217-232(1986)) I.B.R., that of Marck (Nucleic Acids Research 16, 1829-1836 (1988)) I.B.R. or the GCG program of Butler (Methods of Biochemical Analysis 39, 74-97 (1998)) I.B.R.

The new DNA sequence of C. glutamicum which codes for the truB gene and which, as SEQ ID No. 1, is a constituent of the present invention has been found. The amino acid sequence of the corresponding protein has furthermore been derived from the present DNA sequence by the methods described above. The resulting amino acid sequence of the truB gene product is shown in SEQ ID No. 2.

Coding DNA sequences which result from SEQ ID No. 1 by the degeneracy of the genetic code are also a constituent of the invention. In the same way, DNA sequences which hybridize with SEQ ID No. 1 or parts of SEQ ID No. 1 are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in known textbooks of genetics and molecular biology. Amino acid sequences which result in a corresponding manner from SEQ ID No. 2 are also a constituent of the invention.

In the same way, DNA sequences which hybridize with SEQ ID No. 1 or parts of SEQ ID No. 1 are a constituent of the invention. Finally, DNA sequences which are prepared by the polymerase chain reaction (PCR) using primers which result from SEQ ID No. 1 are a constituent of the invention. Such oligonucleotides typically have a length of at least 15 nucleotides.

Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R. The hybridization takes place under stringent conditions, that is to say only hybrids in which the probe and target sequence, i. e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996 I.B.R.).

A 5×SSC buffer at a temperature of approx. 50° C. -68° C., for example, can be employed for the hybridization reaction. Probes can also hybridize here with polynucleotides which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995 I.B.R.) a temperature of approx. 50° C.-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

Instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) I.B.R. and in Newton and Graham: PCR (Spektrum Akademischer Verlag, Heidelberg, Germany, 1994) I.B.R.

It has been found that coryneform bacteria produce amino acids in an improved manner after over-expression of the truB gene.

To achieve an over-expression, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative amino acid production. The expression is likewise improved by measures to prolong the life of the m-RNA. Furthermore, the enzyme activity is also increased by preventing the degradation of the enzyme protein. The genes or gene constructs can either be present in plasmids with a varying number of copies, or can be integrated and amplified in the chromosome. Alternatively, an over-expression of the genes in question can furthermore be achieved by changing the composition of the media and the culture procedure.

Instructions in this context can be found by the expert, inter alia, in Martin et al. (Bio/Technology 5, 137-146 (1987)) I.B.R., in Guerrero et al. (Gene 138, 35-41 (1994)) I.B.R., Tsuchiya and Morinaga (Bio/Technology 6, 428-430 (1988)) I.B.R., in Eikmanns et al. (Gene 102, 93-98 (1991)) I.B.R., in EP 0 472 869 I.B.R., in U.S. Pat. No. 4,601,893 I.B.R., in Schwarzer and Pühler (Bio/Technology 9, 84-87 (1991) I.B.R., in Reinscheid et al. (Applied and Environmental Microbiology 60, 126-132 (1994)) I.B.R., in LaBarre et al. (Journal of Bacteriology 175, 1001-1007 (1993)) I.B.R., in WO 96/15246 I.B.R., in Malumbres et al. (Gene 134, 15-24 (1993)) I.B.R., in JP-A-10-229891 I.B.R., in Jensen and Hammer (Biotechnology and Bioengineering 58, 191-195 (1998)) I.B.R., in Makrides (Microbiological Reviews 60:512-538 (1996)) I.B.R. and in known textbooks of genetics and molecular biology.

By way of example, for enhancement the truB gene according to the invention was over-expressed with the aid of episomal plasmids. Suitable plasmids are those which are replicated in coryneform bacteria. Numerous known plasmid vectors, such as e.g. pZ1 (Menkel et al., Applied and Environmental Microbiology (1989) 64: 549-554 I.B.R.), pEKExl (Eikmanns et al., Gene 102:93-98 (1991) I.B.R.) or pHS2-1 (Sonnen et al., Gene 107:69-74 (1991) I.B.R.) are based on the cryptic plasmids pHM1519, pBL1 or pGA1. Other plasmid vectors, such as e.g. those based on pCG4 (U.S. Pat. No. 4,489,160 I.B.R.), or pNG2 (Serwold-Davis et al., FEMS Microbiology Letters 66, 119-124 (1990) I.B.R.), or pAG1 (U.S. Pat. No. 5,158,891 I.B.R.), can be used in the same manner.

Plasmid vectors which are furthermore suitable are also those with the aid of which the process of gene amplification by integration into the chromosome can be used, as has been described, for example, by Reinscheid et al. (Applied and Environmental Microbiology 60, 126-132 (1994)) I.B.R. for duplication or amplification of the hom-thrB operon. In this method, the complete gene is cloned in a plasmid vector which can replicate in a host (typically E. coli), but not in C. glutamicum. Possible vectors are, for example, pSUP301 (Simon et al., Bio/Technology 1, 784-791 (1983) I.B.R.), pK18mob or pK19mob (Schäfer et al., Gene 145, 69-73 (1994) I.B.R.), pGEM-T (Promega Corporation, Madison, Wis., USA), pCR2.1-TOPO (Shuman (1994). Journal of Biological Chemistry 269:32678-84 I.B.R.; U.S. Pat. No. 5,487,993 I.B.R.), pCR®Blunt (Invitrogen, Groningen, Holland; Bernard et al., Journal of Molecular Biology, 234: 534-541 (1993) I.B.R.), pEMl (Schrumpf et al, 1991, Journal of Bacteriology 173:4510-4516 I.B.R.) or pBGS8 (Spratt et al.,1986, Gene 41: 337-342 I.B.R.). The plasmid vector which contains the gene to be amplified is then transferred into the desired strain of C. glutamicum by conjugation or transformation. The method of conjugation is described, for example, by Schäfer et al. (Applied and Environmental Microbiology 60, 756-759 (1994)) I.B.R. Methods for transformation are described, for example, by Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R. After homologous recombination by means of a “cross over” event, the resulting strain contains at least two copies of the gene in question.

In addition, it may be advantageous for the production of L-amino acids to enhance, in particular over-express one or more enzymes of the particular biosynthesis pathway, of glycolysis, of anaplerosis, of the citric acid cycle, of the pentose phosphate cycle, of amino acid export and optionally regulatory proteins, in addition to the truB gene.

Thus, for the preparation of L-amino acids, in addition to enhancement of the truB gene, one or more endogenous genes chosen from the group consisting of

the dapA gene which codes for dihydrodipicolinate synthase (EP-B 0 197 335 I.B.R.),

the gap gene which codes for glyceraldehyde 3-phosphate dehydrogenase (Eikmanns (1992), Journal of Bacteriology 174:6076-6086 I.B.R.),

the tpi gene which codes for triose phosphate isomerase (Eikmanns (1992), Journal of Bacteriology 174:6076-6086 I.B.R.),

the pgk gene which codes for 3-phosphoglycerate kinase (Eikmanns (1992), Journal of Bacteriology 174:6076-6086 I.B.R.),

the zwf gene which codes for glucose 6-phosphate dehydrogenase (JP-A-09224661 I.B.R.),

the pyc gene which codes for pyruvate carboxylase (DE-A-198 31 609 I.B.R.),

the mqo gene which codes for malate-quinone oxidoreductase (Molenaar et al., European Journal of Biochemistry 254, 395-403 (1998) I.B.R.),

the lysC gene which codes for a feed-back resistant aspartate kinase (Accession No. P26512; EP-B-0387527 I.B.R.; EP-A-0699759 I.B.R.),

the lysE gene which codes for lysine export (DE-A-195 48 222 I.B.R.),

the hom gene which codes for homoserine dehydrogenase (EP-A 0131171 I.B.R.),

the ilvA gene which codes for threonine dehydratase (Möckel et al., Journal of Bacteriology (1992) 8065-8072) I.B.R.) or the ilvA(Fbr) allele which codes for a “feed back resistant” threonine dehydratase (Möckel et al., (1994) Molecular Microbiology 13: 833-842 I.B.R.),

the ilvBN gene which codes for acetohydroxy-acid synthase (EP-B 0356739 I.B.R.),

the ilvD gene which codes for dihydroxy-acid dehydratase (Sahm and Eggeling (1999) Applied and Environmental Microbiology 65: 1973-1979 I.B.R.),

the zwa1 gene which codes for the Zwa1 protein (DE: 19959328.0 I.B.R., DSM 13115),

Can be enhanced, in particular over-expressed.

It may furthermore be advantageous for the production of L-amino acids, in addition to the enhancement of the truB gene, for one or more genes chosen from the group consisting of:

the pck gene which codes for phosphoenol pyruvate carboxykinase (DE 199 50 409.1 I.B.R.; DSM 13047),

the pgi gene which codes for glucose 6-phosphate isomerase (U.S. Ser. No. 09/396,478 I.B.R.; DSM 12969),

the poxB gene which codes for pyruvate oxidase (DE: 1995 1975.7 I.B.R.; DSM 13114),

the zwa2 gene which codes for the Zwa2 protein (DE: 19959327.2 I.B.R., DSM 13113)

To be attenuated, in particular for the expression thereof to be reduced.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme with a low activity or inactivates the corresponding gene or enzyme (protein), and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

In addition to over-expression of the truB gene it may furthermore be advantageous for the production of amino acids to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982 I.B.R.).

The invention also provides the microorganisms prepared according to the invention, and these can be cultured continuously or discontinuously in the batch process (batch culture) or in the fed batch (feed process) or repeated fed batch process (repetitive feed process) for the purpose of production of amino acids. A summary of known culture methods is described in the textbook by Chmiel (Bioprozesstechnik 1. Einführung in die Bioverfahrenstechnik [Bioprocess Technology 1. Introduction to Bioprocess Technology] (Gustav Fischer Verlag, Stuttgart, 1991)) I.B.R. or in the textbook by Storhas (Bioreaktoren und periphere Einrichtungen [Bioreactors and Peripheral Equipment] (Vieweg Verlag, Braunschweig/Wiesbaden, 1994)) I.B.R.

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981) I.B.R.

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e. g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

Methods for the determination of L-amino acids are known from the prior art. The analysis can thus be carried out, for example, as described by Spackman et al. (Analytical Chemistry, 30, (1958), 1190 I.B.R.) by ion exchange chromatography with subsequent ninhydrin derivation, or it can be carried out by reversed phase HPLC, for example as described by Lindroth et al. (Analytical Chemistry (1979) 51: 1167-1174) I.B.R.

The process according to the invention is used for fermentative preparation of amino acids.

The following microorganism was deposited as a pure culture on Aug. 22, 2001 at the Deutsche Sammlung für Mikroorganismen und Zellkulturen (DSMZ=German Collection of Microorganisms and Cell Cultures, Braunschweig, Germany) in accordance with the Budapest Treaty:

Escherichia coli DH5alphamcr/pEC-XK99EtruBex (=DH5αmcr/pEC-XK99EtruBex) as DSM 14460.

The present invention is explained in more detail in the following with the aid of embodiment examples.

The isolation of plasmid DNA from Escherichia coli and all techniques of restriction, Klenow and alkaline phosphatase treatment were carried out by the method of Sambrook et al. (Molecular Cloning. A Laboratory Manual (1989) Cold Spring Harbor Laboratory Press, Cold Spring Harbor, N.Y., USA) I.B.R. Methods for transformation of Escherichia coli are also described in this handbook.

The composition of the usual nutrient media, such as LB or TY medium, can also be found in the handbook by Sambrook et al.

### Example 1

Preparation of a Genomic Cosmid Gene Library From Corynebacterium glutamicum ATCC 13032

Chromosomal DNA from Corynebacterium glutamicum ATCC 13032 was isolated as described by Tauch et al. (1995, Plasmid 33:168-179) I.B.R. and partly cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Diagnostics GmbH, Mannheim, Germany, Product Description SAP, Code no. 1758250). The DNA of the cosmid vector SuperCos1 (Wahl et al. (1987) Proceedings of the National Academy of Sciences USA 84:2160-2164 I.B.R.), obtained from Stratagene (La Jolla, USA, Product Description SuperCos1 Cosmid Vector Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no. 27-0870-04). The ligation mixture was then packed in phages with the aid of Gigapack II XL Packing Extract (Stratagene, La Jolla, USA, Product Description Gigapack II XL Packing Extract, Code no. 200217).

For infection of the E. coli strain NM554 (Raleigh et al. 1988, Nucleic Acids Research 16:1563-1575 I.B.R.) the cells were taken up in 10 mM MgSO₄ and mixed with an aliquot of the phage suspension. The infection and titering of the cosmid library were carried out as described by Sambrook et al. (1989, Molecular Cloning: A Laboratory Manual, Cold Spring Harbor) I.B.R., the cells being plated out on LB agar (Lennox, 1955, Virology, 1:190) I.B.R. with 100 mg/l ampicillin. After incubation overnight at 37° C., recombinant individual clones were selected.

### Example 2

Isolation and Sequencing of the truB Gene

The cosmid DNA of an individual colony was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Diagnostics GmbH, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany).

The DNA of the sequencing vector pzero-1, obtained from Invitrogen (Groningen, Holland, Product Description Zero Background Cloning Kit, Product No. K2500-01), was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A Laboratory Manual, Cold Spring Harbor) I.B.R., the DNA mixture being incubated overnight with T4 ligase (Pharmacia Biotech, Freiburg, Germany). This ligation mixture was then electroporated (Tauch et al. 1994, FEMS Microbiol Letters, 123:343-7) I.B.R. into the E. coli strain DH5αMCR (Grant, 1990, Proceedings of the National Academy of Sciences U.S.A., 87:4645-4649 I.B.R.) and plated out on LB agar (Lennox, 1955, Virology, 1:190 I.B.R.) with 50 mg/l zeocin.

The plasmid preparation of the recombinant clones was carried out with the Biorobot 9600 (Product No. 900200, Qiagen, Hilden, Germany). The sequencing was carried out by the dideoxy chain termination method of Sanger et al. (1977, Proceedings of the National Academy of Sciences U.S.A., 74:5463-5467) I.B.R. with modifications according to Zimmermann et al. (1990, Nucleic Acids Research, 18:1067) I.B.R. The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231 I.B.R.) version 97-0. The individual sequences of the pzerol derivatives were assembled to a continuous contig. Further analyses can be carried out with the “BLAST search program” (Altschul et al., 1997, Nucleic Acids Research, 25:3389-3402 I.B.R.) against the non-redundant databank of the “National Center for Biotechnology Information” (NCBI, Bethesda, Md., USA) I.B.R.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.

The resulting nucleotide sequence is shown in SEQ ID No. 1. Analysis of the nucleotide sequence showed an open reading frame of 894 base pairs, which was called the truB gene. The truB gene codes for a protein of 297 amino acids.

### Example 3

Preparation of a Shuttle Vector pEC-XK99EtruBex for Enhancement of the truB gene in C. glutamicum

3.1 Cloning of the truB Gene in the Vector pCR®Blunt II

From the strain ATCC 13032, chromosomal DNA was isolated by the method of Eikmanns et al. (Microbiology 140: 1817-1828 (1994)) I.B.R. On the basis of the sequence of the truB gene known for C. glutamicum from example 2, the following oligonucleotides were chosen for the polymerase chain reaction (see also SEQ ID No. 3 and SEQ ID No. 4):

truBex1:

5′-tg ggtacc-acg gca tag act caa cag ac-3′ SEQ ID NO: 3

truBex2:

5′-ga tctaga-cgg ctt ctg gca gac caa ct-3′ SEQ ID NO: 4

The primers shown were synthesized by MWG-Biotech AG (Ebersberg, Germany) and the PCR reaction was carried out by the standard PCR method of Innis et al. (PCR Protocols. A Guide to Methods and Applications, 1990, Academic Press) I.B.R. with Pwo-Polymerase from Roche Diagnostics GmbH (Mannheim, Germany). With the aid of the polymerase chain reaction, the primers allow amplification of a DNA fragment 948 bp in size, which carries the truB gene. Furthermore, the primer truBex1 contains the sequence for the cleavage site of the restriction endonuclease Kpnl, and the primer truBex2 the cleavage site of the restriction endonuclease XbaI, which are marked by underlining in the nucleotide sequence shown above.

The truB fragment 948 bp in size was cleaved with the restriction endonucleases KpnI and XbaI and then isolated from the agarose gel with the QiaExII Gel Extraction Kit (Product No. 20021, Qiagen, Hilden, Germany).

3.2 Construction of the Shuttle Vector pEC-XK99E

The E. coli-C. glutamicum shuttle vector pEC-XK99E was constructed according to the prior art. The vector contains the replication region rep of the plasmid pGA1 including the replication effector per (U.S. Pat. No. 5,175,108 I.D.R.; Nesvera et al., Journal of Bacteriology 179, 1525-1532 (1997) I.B.R.), the kanamycin resistance gene aph(3′)-IIa from Escherichia coli (Beck et al. (1982), Gene 19: 327-336 I.B.R.), the replication origin of the trc promoter, the termination regions T1 and T2, the lacI^{q} gene (repressor of the lac operon of E. coli) and a multiple cloning site (mcs) (Norrander, J. M. et al. Gene 26, 101-106 (1983) I.B.R.) of the plasmid pTRC99A (Amann et al. (1988), Gene 69: 301-315 I.B.R.).

The trc promoter can be induced by addtion of the lactose derivative IPTG (isopropyl β-D-thiogalactopyranoside).

The E. coli-C. glutamicum shuttle vector pEC-XK99E constructed was transferred into C. glutamicum DSM5715 by means of electroporation (Liebl et al., 1989, FEMS Microbiology Letters, 53:299-303 I.B.R.). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 25 mg/l kanamycin. Incubation was carried out for 2 days at 33° C.

Plasmid DNA was isolated from a transformant by conventional methods (Peters-Wendisch et al., 1998, Microbiology, 144, 915-927 I.B.R.), cleaved with the restriction endonuclease HindIII, and the plasmid was checked by subsequent agarose gel electrophoresis.

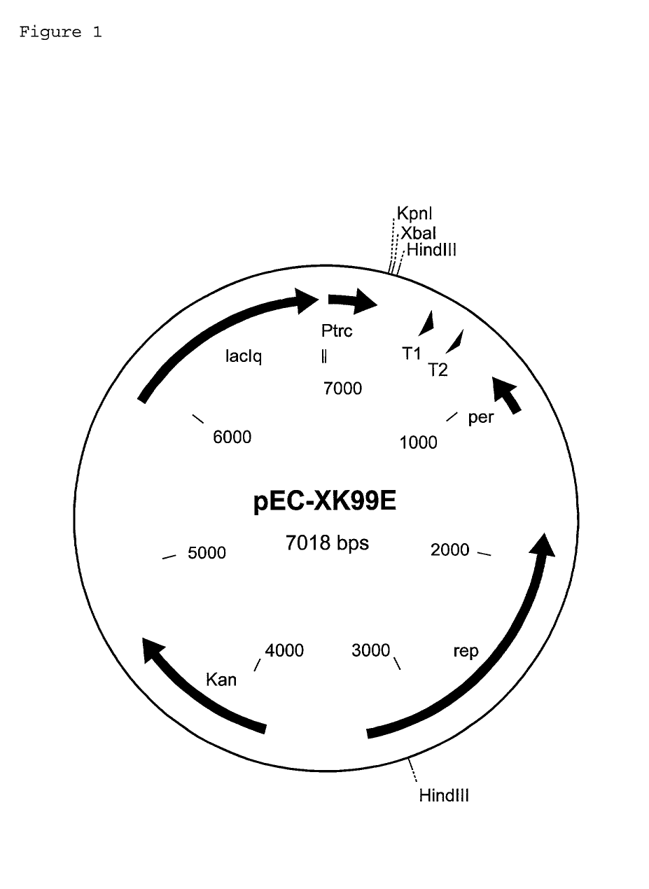

The plasmid construct obtained in this way was called pEC-XK99E (FIG. 1). The strain obtained by electroporation of the plasmid pEC-XK99E in the C. glutamicum strain DSM5715 was called DSM5715/pEC-XK99E and deposited as DSM13455 at the Deutsche Sammlung fur Mikroorganismen und Zellkulturen (DSMZ=German Collection of Microorganisms and Cell Cultures, Braunschweig, Germany) in accordance with the Budapest Treaty.

3.3 Cloning of truB in the E. coli-C. glutamicum Shuttle Vector pEC-XK99E

The E. coli-C. glutamicum shuttle vector pEC-XK99E described in example 3.2 was used as the vector. DNA of this plasmid was cleaved completely with the restriction enzymes KpnI and XbaI and then dephosphorylated with shrimp alkaline phosphatase (Roche Diagnostics GmbH, Mannheim, Germany, Product Description SAP, Product No. 1758250).

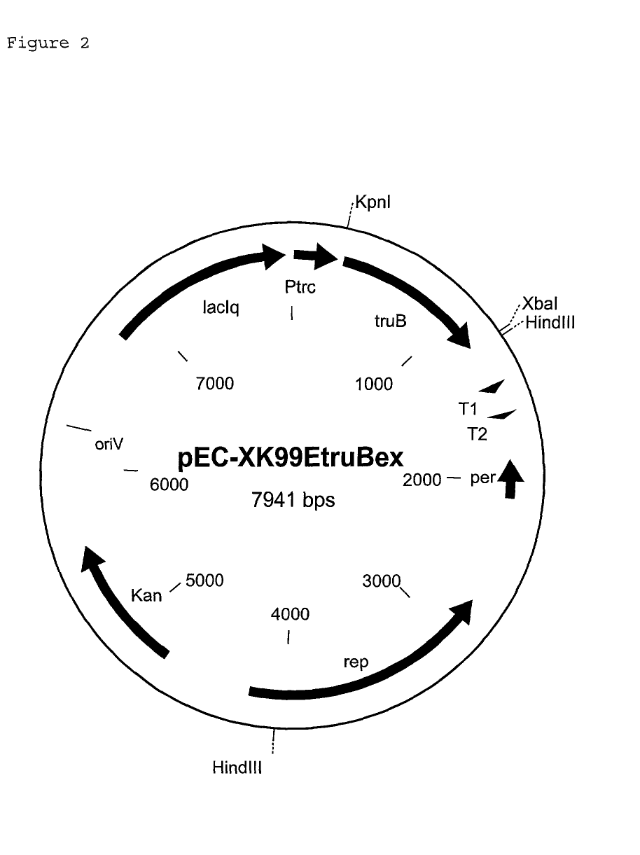

The truB fragment approx. 930 bp in size described in example 3.1, obtained by means of PCR and cleaved with the restriction endonucleases KpnI and XbaI was mixed with the prepared vector pEC-XK99E and the batch was treated with T4 DNA ligase (Amersham Pharmacia, Freiburg, Germany, Product Description T4-DNA-Ligase, Code no. 27-0870-04). The ligation batch was transformed in the E. coli strain DH5αmcr (Hanahan, In: DNA Cloning. A Practical Approach. Vol. I, IRL-Press, Oxford, Washington DC, USA) I.B.R. Selection of plasmid-carrying cells was made by plating out the transformation batch on LB agar (Lennox, 1955, Virology, 1:190 I.B.R.) with 50 mg/l kanamycin. After incubation overnight at 37° C., recombinant individual clones were selected. Plasmid DNA was isolated from a transformant with the Qiaprep Spin Miniprep Kit (Product No. 27106, Qiagen, Hilden, Germany) in accordance with the manufacturer's instructions and cleaved with the restriction enzymes XbaI and KpnI to check the plasmid by subsequent agarose gel electrophoresis. The resulting plasmid was called pEC-XK99EtruBex. It is shown in FIG. 2.

This application claims priority to German Priority Document Application No. 100 47 864.6, filed on Sep. 27, 2000. The above German Priority Document is hereby incorporated by reference in its entirety.

We claim:

1. An isolated polynucleotide from coryneform bacteria, comprising a polynucleotide sequence which codes for the truB gene, selected from the group consisting of:
a) polynucleotide which is identical to the extent of at least 70% to a polynucleotide which codes for a polypeptide which comprises the amino acid sequence of SEQ ID No. 2,
b) polynucleotide which codes for a polypeptide which comprises an amino acid sequence which is identical to the extent of at least 70% to the amino acid sequence of SEQ ID No. 2,
c) polynucleotide which is complementary to the polynucleotides of a) or b), and
d) polynucleotide comprising at least 15 successive nucleotides of the polynucleotide sequence of a), b) or c).

2. The polynucleotide according to claim 1, wherein the polypeptide has tRNA pseudouridine 55 synthase activity.

3. The polynucleotide according to claim 1, wherein the polynucleotide is a recombinant DNA which is capable of replication in coryneform bacteria.

4. The polynucleotide according to claim 1, wherein the polynucleotide is an RNA.

5. The polynucleotide according to claim 3, comprising the nucleic acid sequence as shown in SEQ ID No. 1.

6. The polynucleotide according to claim 3, wherein the DNA, comprises
(i) the nucleotide sequence shown in SEQ ID No. 1, or
(ii) at least one sequence which corresponds to sequence (i) within the range of the degeneration of the genetic code, or
(iii) at least one sequence which hybridizes with the sequence complementary to sequence (i) or (ii).

7. The polynucleotide according to claim 6, further comprising
(iv) sense mutations of neutral function in (i).

8. The polynucleotide according to claim 6, wherein the hybridization of sequence (iii) is carried out under conditions of stringency corresponding at most to 2x SSC.

9. A polynucleotide sequence according to claim 1, wherein the polynucleotide codes for a polypeptide that comprises the amino acid sequence shown in SEQ ID NO: 2.

10. A coryneform bacteria in which the truB gene is enhanced.

11. The coryneform bacteria according to claim 10, wherein the truB gene is over-expressed.

12. An Escherichia coli strain DH5alphamcr/pEC-XK99EtruBex deposited as DSM 14460.

13. A method for the fermentative preparation of L-amino acids in coryneform bacteria, comprising:
a) fermenting, in a medium, the coryneform bacteria which produce the desired L-amino acid and in which at least the endogenous truB gene or nucleotide sequences which code for it are enhanced.

14. The method according to claim 13, further comprising:
b) concentrating the L-amino acid in the medium or in the cells of the bacteria.

15. The method according to claim 14, further comprising:
c) isolating the L-amino acid.

16. The method according to claim 13, wherein the L amino acids are lysine.

17. The method according to claim 13, wherein truB gene or nucleotide sequences coding for this gene are overexpressed.

18. The method according to claim 13, wherein additional genes of the biosynthesis pathway of the desired L-amino acid are enhanced in the bacteria.

19. The method according to claim 13, wherein bacteria in which the metabolic pathways which reduce the formation of the desired L-amino acid are at least partly eliminated are employed.

20. The method according to claim 13, wherein a strain transformed with a plasmid vector is employed, and the plasmid vector carries the nucleotide sequence which codes for the truB gene.

21. The method according to claim 13, wherein the expression of the polynucleotide(s) which code(s) for the truB gene is enhanced.

22. The method according to claim 21, wherein the expression of the polynucleotide(s) which code(s) for the truB gene is over-expressed.

23. The method according to claim 13, wherein the catalytic properties of the polypeptide for which the polynucleotide truB codes are increased.

24. The method according to claim 13, wherein the bacteria being fermented comprise, at the same time, one or more genes which are enhanced or overexpressed; wherein the one or more genes is/are selected from the group consisting of:
the dapA gene which codes for dihydrodipicolinate synthase,
the gap gene which codes for glyceraldehyde 3-phosphate dehydrogenase,
the tpi gene which codes for triose phosphate isomerase,
the pgk gene which codes for 3-phosphoglycerate kinase,
the zwf gene which codes for glucose 6-phosphate dehydrogenase,
the pyc gene which codes for pyruvate carboxylase,
the mqo gene which codes for malate-quinone oxidoreductase,
the lysC gene which codes for a feed-back resistant aspartate kinase,
the lysE gene which codes for lysine export,
the hom gene which codes for homoserine dehydrogenase
the ilvA gene which codes for threonine dehydratase or the ilvA(Fbr) allele which codes for a feed back resistant threonine dehydratase,
the ilvBN gene which codes for acetohydroxy-acid synthase,
the ilvD gene which codes for dihydroxy-acid dehydratase, and
the zwa1 gene which codes for the Zwa1 protein.

25. The method according to claim 13, wherein the bacteria being fermented comprise, at the same time, one or more genes which are attenuated; wherein the genes are selected from the group consisting of:
the pck gene which codes for phosphoenol pyruvate carboxykinase,
the pgi gene which codes for glucose 6-phosphate isomerase,
the poxB gene which codes for pyruvate oxidase, and the zwa2 gene which codes for the Zwa2 protein.

26. The method according to claim 13, wherein microorganisms of the species Corynebacterium glutamicum are employed.

27. A coryneform bacteria, comprising a vector which carries a polynucleotide according to claim 1.

28. A method for discovering RNA, cDNA and DNA in order to isolate nucleic acids or polynucleotides or genes which code for tRNA pseudouridine 55 synthase or have a high similarity with the sequence of the truB gene, comprising contacting the RNA, cDNA, or DNA with hybridization probes comprising polynucleotide sequences according to claim 1.

29. The method according to claim 28, wherein arrays, micro arrays or DNA chips are employed.
